(19) United States
(12) Patent Application Publication
(10) Pub. No.: US 2014/0364718 A1
(43) Pub. Date: Dec. 11, 2014

(54) MEDICAL IMAGE DIAGNOSTIC APPARATUS BED AND MEDICAL IMAGE DIAGNOSTIC APPARATUS

(71) Applicants: Kabushiki Kaisha Toshiba, Minato-ku (JP); Toshiba Medical Systems Corporation, Otawara-shi (JP)

(72) Inventor: Manabu TESHIGAWARA, Otawara (JP)

(73) Assignees: KABUSHIKI KAISHA TOSHIBA, Minato-ku (JP); Toshiba Medical Systems Corporation, Otawara-shi (JP)

(21) Appl. No.: 14/466,050

(22) Filed: Aug. 22, 2014

Related U.S. Application Data
(63) Continuation of application No. PCT/JP2013/078739, filed on Oct. 23, 2013.

(30) Foreign Application Priority Data
Oct. 24, 2012 (JP) 2012-234619
Oct. 22, 2013 (JP) 2013-219670

(51) Int. Cl. A61G 7/018; A61B 6/00; A61B 6/03; A61B 6/04; A61G 7/005; A61B 5/055

(52) U.S. Cl. CPC A61G 7/018; A61G 7/005; A61B 5/0555; A61B 6/032; A61B 6/037; A61B 6/0407; A61B 6/4417
USPC 600/407; 5/610

(57) ABSTRACT

A medical image diagnostic apparatus bed according to an embodiment includes a top, a support mechanism, a determination unit, and a driving unit. On the top, a subject is placed. The support mechanism supports the top with adjustability of an elevation angle of the top. The determination unit determines the elevation angle based on information concerning deflection of the top. The driving unit drives the support mechanism in accordance with the determined elevation angle.

### CROSS-REFERENCE TO RELATED APPLICATIONS

This application is a Continuation application of PCT Application No. PCT/JP2013/078739, filed Oct. 23, 2013 and based upon and claims the benefit of priority from the Japanese Patent Application No. 2012-234619, filed Oct. 24, 2012, and the Japanese Patent Application No. 2013-219670, filed Oct. 22, 2013, the entire contents of all of which are incorporated herein by reference.

### FIELD

Embodiments described herein relate generally to a medical image diagnostic apparatus bed and a medical image diagnostic apparatus.

### BACKGROUND

A medical image diagnostic apparatus including a scanning unit is configured to move a subject placed on the top of the bed into the field of view (to be referred to as the FOV hereinafter) of the scanning unit. Such medical image diagnostic apparatuses include an X-ray computed tomography apparatus (to be referred to as an X-ray CT apparatus hereinafter), a single photon emission computed tomography apparatus (to be referred to as a SPECT apparatus hereinafter), a positron emission computed tomography apparatus (to be referred to as a PET apparatus hereinafter), and a magnetic resonance imaging apparatus (to be referred to as an MRI apparatus hereinafter).

At this time, the top deflects in accordance with a feeding amount by which the top is fed from the bed. The position of a subject in the FOV changes in the long-axis direction of the top due to the deflection of the top (or to be also referred to as top sag). In a scan scheme called step & shoot (to be referred to as S & S hereinafter), for example, discontinuous differences in level occur on a reconstructed image (sagittal image) for each imaging region (bed). Discontinuous differences in level in imaging by S & S scanning originate from differences in the feeding length of the top in the respective steps. That is, the top sinks differently for each step, which causes discontinuous differences in level at joints between images.

FIG. 8 is a view showing an example of the positional relationship between the top and imaging regions in S & S scanning in a PET apparatus. As shown in FIG. 8, as the feeding amount of the top from the bed increases, the top sag in an imaging region increases. At this time, the position of the top sinks from a horizontal state.

FIG. 9 is a view showing an example of the image obtained by concatenating the reconstructed images, reconstructed from a plurality of imaging regions in FIG. 8, along the long-axis direction of the top. FIG. 9 shows the differences in level of the top on the image (PET sagittal image) obtained by concatenating the reconstructed images respectively corresponding to imaging regions 1 to 3. In addition, FIG. 9 shows the top surface in each imaging region in the form of an oblique line segment. In imaging region 1 in FIG. 9 in which the feeding amount of the top is large, the tilt of the top is large and the deflection angle is large. In imaging region 3 in FIG. 9 in which the feeding amount of the top is small, the tilt of the top is small and the deflection angle is small. In either case, the tilt and difference in level of the top appear on the image obtained by concatenating reconstructed images.

In addition, FIG. 10 is a view showing an example of the top surface on a sagittal image (to be referred to as a helical sagittal image hereinafter) reconstructed by continuous top moving imaging (to be referred to as helical scanning hereinafter) by an X-ray CT apparatus or the like. Helical scanning is an imaging method in which the feeding amount of the top is continuously changed. FIG. 10 is a view showing an example of the top surface on a helical sagittal image in helical scanning. In this case, a top surface is obtained as a curve, with the length of the long axis of an FOV being set to 0 as a limit. A top curve representing a top is continuous. However, for example, positioning with images in radiotherapy apparatuses having horizontal tops will produce an insufficient result. For example, as is obvious from FIGS. 9 and 10, the problem of the positional shift of the top occurs on the superimposed image obtained by superimposing the PET sagittal image reconstructed by S & S scanning in a PET apparatus on a helical sagittal image.

In an apparatus as a combination of a plurality of modalities such as a PET/CT apparatus, PET/MRI apparatus, and SPECT/CT apparatus, a mechanism for preventing the above discontinuous differences in level (to be referred to as top sag level differences hereinafter) is especially important when superimposing the medical images generated by a plurality of modalities. Assume that a subject is placed on a horizontal top at the time of imaging by an X-ray CT apparatus, PET apparatus, or the like in a radiotherapy plan. In this case, it is easy to position the reconstructed images reconstructed by the X-ray CT apparatus, the PET apparatus, and the like. However, the problem of the deflection of the top due to the weight of the subject exists.

As a measure to solve this problem, the rigidity of the top may be improved. Even if, however, the rigidity of the top is improved, the problem of top sag is unavoidable to various degrees in principle. As another measure to solve the above problem, columns, rails, or the like which support the top are sometimes provided within the field of view of a detector. However, these measures influence the absorption of X-rays and gamma-rays and hence become causes of artifacts.

### BRIEF DESCRIPTION OF THE DRAWINGS

FIG. 1 is a block diagram showing an example of the arrangement of a medical image diagnostic apparatus bed according to an embodiment.

FIG. 2 is a flowchart showing an example of a procedure for vertical direction moving operation according to this embodiment.

FIG. 3 is a view showing an example of driving a vertical direction moving mechanism upward in the vertical direction in accordance with a vertical direction moving amount according to this embodiment.

FIG. 4 is a view showing an example of translating a top downward in the vertical direction in accordance with a translation amount according to this embodiment.

FIG. 5 is a block diagram showing an example of the arrangement of a medical image diagnostic apparatus including a medical image diagnostic apparatus bed according to a modification of this embodiment.

FIG. 6 is a flowchart showing an example of a procedure for vertical direction moving operation according to the modification of this embodiment.

FIG. 7 is a view showing an example of the image obtained by concatenating the reconstructed images, reconstructed from a plurality of imaging regions, along the long-axis direction of the top according to the modification of this embodiment.

FIG. 8 is a view showing an example of the positional relationship between the top and imaging regions according to conventional S & S scanning in a PET.

FIG. 9 is a view showing an example of the image obtained by concatenating the reconstructed images, reconstructed from a plurality of imaging regions in FIG. 8, along the long-axis direction of the top.

FIG. 10 is a view showing an example of the top surface on the sagittal image reconstructed in continuous top moving imaging by a conventional X-ray computed tomography apparatus.

### DETAILED DESCRIPTION

A medical image diagnostic apparatus bed according to embodiment includes a top, a support mechanism, a determination unit, and a driving unit. On the top, a subject is placed. The support mechanism supports the top with adjustability of an elevation angle of the top. The determination unit determines the elevation angle based on information concerning deflection of the top. The driving unit drives the support mechanism in accordance with the determined elevation angle.

An embodiment of a medical image diagnostic apparatus bed will be described below with reference to the accompanying drawings. Medical image diagnostic apparatuses to which this medical image diagnostic apparatus bed can be applied include, for example, an X-ray computed tomography apparatus (to be referred to as an X-ray CT apparatus hereinafter), X-ray diagnostic apparatus, magnetic resonance imaging apparatus (to be referred to as an MRI apparatus hereinafter), nuclear medicine diagnostic apparatus (positron emission computed tomography apparatus (to be referred to as a PET apparatus hereinafter), single photon emission computed tomography apparatus (to be referred to as a SPECT apparatus hereinafter)), PET/CT apparatus, SPECT/CT apparatus, PET/MRI apparatus, and SPECT/MRI apparatus. An example of applying this embodiment to an X-ray CT apparatus and an X-ray diagnostic apparatus, as an example of applying the embodiment to a medical image diagnostic apparatus, will be described later as a modification of the embodiment. Note that the same reference numerals in the following description denote constituent elements having almost the same functions and arrangements, and a repetitive description will be made only when required.

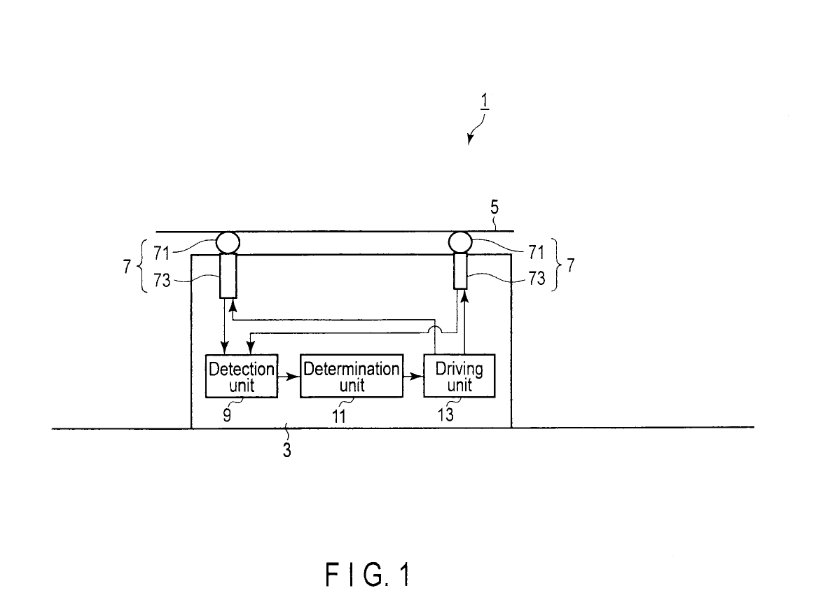

FIG. 1 is a block diagram showing an example of the arrangement of a medical image diagnostic apparatus bed 1 according to this embodiment. The medical image diagnostic apparatus bed 1 according to the embodiment includes a bed main body 3, a top 5, and a plurality of support mechanisms 7 which support the top 5 at a plurality of fulcrums. Note that the number of support mechanisms 7 may be one. The bed main body 3 includes a detection unit 9, a determination unit 11, and a driving unit 13. The support mechanism 7 includes a long-axis direction moving mechanism 71 which supports the top 5 with its movability in the long-axis direction of the top 5 and a vertical direction moving mechanism 73 which supports the top 5 and the long-axis direction moving mechanism 71 with their movability in the vertical direction.

A subject is placed on the top 5. The plurality of support mechanisms 7 respectively having a plurality of fulcrums (to be described later) support the top 5. The long-axis direction moving mechanisms 71 (to be described later) support the top with its movability in the long-axis direction. The vertical direction moving mechanisms 73 (to be described later) support the top 5 and the long-axis direction moving mechanisms 71 with movability of the top 5 in the vertical direction through the long-axis direction moving mechanisms 71.

Each of the support mechanisms 7 has a fulcrum to support the top 5. Each of the support mechanisms 7 includes the long-axis direction moving mechanism 71 and the vertical direction moving mechanism 73. The long-axis direction moving mechanism 71 includes a rotating body for feeding the top 5 in the long-axis direction. The rotating body is, for example, a roller having a rotation axis in the short-axis direction of the top 5. The long-axis direction moving mechanism 71 moves the top 5 in the long-axis direction by rotating the rotating body in accordance with driving by the driving unit 13 (to be described later).

The vertical direction moving mechanism 73 has a linear motion bearing (not shown) which supports the long-axis direction moving mechanism 71 with its movability in the vertical direction. The vertical direction moving mechanisms 73 move the long-axis direction moving mechanisms 71 and the top 5 in the vertical direction along the linear motion bearings in accordance with driving by the driving unit 13 (to be described later).

The detection unit 9 detects information concerning the deflection of the top 5. Information concerning deflection includes, for example, the weight of the subject placed on the top 5, the moving amount (to be referred to as the long-axis direction moving amount hereinafter) of the top 5 moved by the long-axis direction moving mechanisms 71 in the long-axis direction, and the deflection amount of the top 5.

More specifically, the detection unit 9 detects the weight of the subject placed on the top 5 via the support mechanisms 7. The detection unit 9 detects a long-axis direction moving amount from the rotation of each rotating body. The detection unit 9 outputs the detected weight and long-axis direction moving amount to the determination unit 11 (to be described later). More specifically, the detection unit 9 detects the weight applied to each of the support mechanisms 7 in accordance with the movement of the top 5, on which the subject is placed, in the long-axis direction. The detection unit 9 outputs the weight applied to each of the support mechanisms 7, which corresponds to a long-axis direction moving amount, to the determination unit 11 (to be described later).

Note that a plurality of detection units 9 may be arranged below the lower surface of the top 5. In this case, the detection units 9 detect the distribution of the weights of the subject which are applied to the top 5. The weight distribution is the weight distribution of the subject along the long-axis direction. Note that the detection units 9 may detect the weight distribution of the subject along the long-axis direction and a change in weight distribution along with the movement of the top 5, on which the subject is placed, in the long-axis direction. In addition, the detection units 9 may detect the deflection amount of the top 5 by using, for example, electromagnetic waves.

In addition, the detection unit 9 may detect the placement position (e.g., the center of gravity) of the subject on the top 5. The detection unit 9 outputs the detected placement position of the subject to the determination unit 11 (to be described later).

The determination unit 11 determines the deflection amount of the top based on the long-axis direction moving amount and weight output from the detection unit 9. The determination unit 11 determines the elevation angle of the top 5 based on the deflection amount. The elevation angle is determined to make part of the top 5 moved in the long-axis direction become horizontal. Part of the top 5 is, for example, the top 5 moved in a field of view (to be referred to as an FOV hereinafter) (not shown). More specifically, the determination unit 11 stores the first correspondence table of deflection amounts corresponding to long-axis direction moving amounts and weights. The determination unit 11 stores the second correspondence table of elevation angles corresponding to deflection amounts. The determination unit 11 determines a deflection amount by using the first correspondence table, a long-axis direction moving amount, and a weight. The determination unit 11 determines an elevation angle by using the second correspondence table and the deflection amount. Note that the determination unit 11 may determine a deflection amount based on a weight distribution and a long-axis direction moving amount.

The determination unit 11 may also store the third correspondence table (the correspondence table of elevation angles corresponding to long-axis direction moving amounts and weights) as a combination of the first correspondence table and the second correspondence table. In this case, the determination unit 11 determines an elevation angle based on a long-axis direction moving amount and a weight. Note that the determination unit 11 may determine the weight distribution of the subject in the long-axis direction based on the weight of the subject detected by moving the top 5, on which the subject is placed, in the long-axis direction and the weight of the subject input in advance. In addition, the determination unit 11 may calculate the weight distribution of the subject along the long-axis direction based on a detected weight and the weight and height of the subject input in advance.

The determination unit 11 determines a moving amount (to be referred to as a vertical direction moving amount hereinafter) along the vertical direction with respect to at least one of the plurality of support mechanisms 7. When, for example, the number of support mechanisms 7 is two as shown in FIG. 1, a determined vertical direction moving amount concerns a support mechanism near a gantry (not shown). The determination unit 11 outputs vertical direction moving amounts corresponding to the respective support mechanisms 7 to the driving unit 13 (to be described later). Note that the determination unit 11 may determine vertical direction moving amounts based on the top curve obtained by scanography.

The determination unit 11 determines the translation amounts by which the plurality of support mechanisms 7 are translated along the vertical direction, based on the determined elevation angle. For example, the determination unit 11 determines translation amounts so as to make part of the top 5 in the FOV coincide with the position of the top surface in the vertical direction. The determination unit 11 outputs the determined translation amounts to the driving unit 13 (to be described later). For example, the determination unit 11 determines translation amounts so as to place part of the top 5 at a predetermined position in the FOV in a scan scheme (to be referred to as S & S scanning hereinafter) called step & shoot (to be referred to as S & S hereinafter).

The determination unit 11 may also calculate moments around the fulcrums which support the top 5 based on a weight distribution and a long-axis direction moving amount. In this case, the determination unit 11 calculates the deflection amount of the top 5 based on the calculated moments. The determination unit 11 then determines an elevation angle based on the calculated deflection amount. Note that the determination unit 11 may calculate moments around the fulcrums which support the top 5 based on the placement position (center of gravity) of the subject, the weight of the subject, and the positions of the fulcrums.

The determination unit 11 also includes a memory (not shown). The memory stores information of a subject (patient information) and an imaging plan for the subject. Note that a storage unit (not shown) may store the patient information and the imaging plan. The determination unit 11 calculates the deflection amount of the top 5 based on the patient information and the imaging plan. More specifically, the determination unit 11 specifies the height and weight of the subject based on the patient information. The determination unit 11 specifies a region of the subject placed the distal end side of the top in the moving direction based on the imaging plan. This region is for example, the head portion (head first) or foot portion (foot first) of the subject. Note that the determination unit 11 may determine the above region based on the placement position (center of gravity) of the subject on the top 5. The determination unit 11 may estimate the deflection amount of the top 5 which corresponds to the long-axis direction moving amount based on the height and weight of the subject and “head first” or “foot first”. In this case, the determination unit 11 determines an elevation angle based on the estimated deflection amount.

The driving unit 13 drives the support mechanisms 7. More specifically, the driving unit 13 drives the long-axis direction moving mechanisms 71 to feed the top 5 in the long-axis direction in accordance with an instruction from an input unit (not shown). With this driving operation, the top 5 moves along the long-axis direction. The driving unit 13 drives each of the vertical direction moving mechanisms 73 of the support mechanisms 7 in accordance with the vertical direction moving amount output from the determination unit 11. With this driving operation, part of the top 5 in the FOV becomes horizontal.

The driving unit 13 drives the plurality of vertical direction moving mechanisms 73 to move the vertical direction moving mechanisms 73 of the support mechanisms 7 along the vertical direction in accordance with the translation amount output from the determination unit 11. With this driving operation, part of the top 5 in the FOV always remains the same height.

### (Vertical Direction Moving Function)

The vertical direction moving function is a function of moving the vertical direction moving mechanisms 73 in the vertical direction based on the vertical direction moving amount and translation amount determined by the determination unit 11. Operation concerning the vertical direction moving function (to be referred to as vertical direction moving operation hereinafter) is described hereinafter.

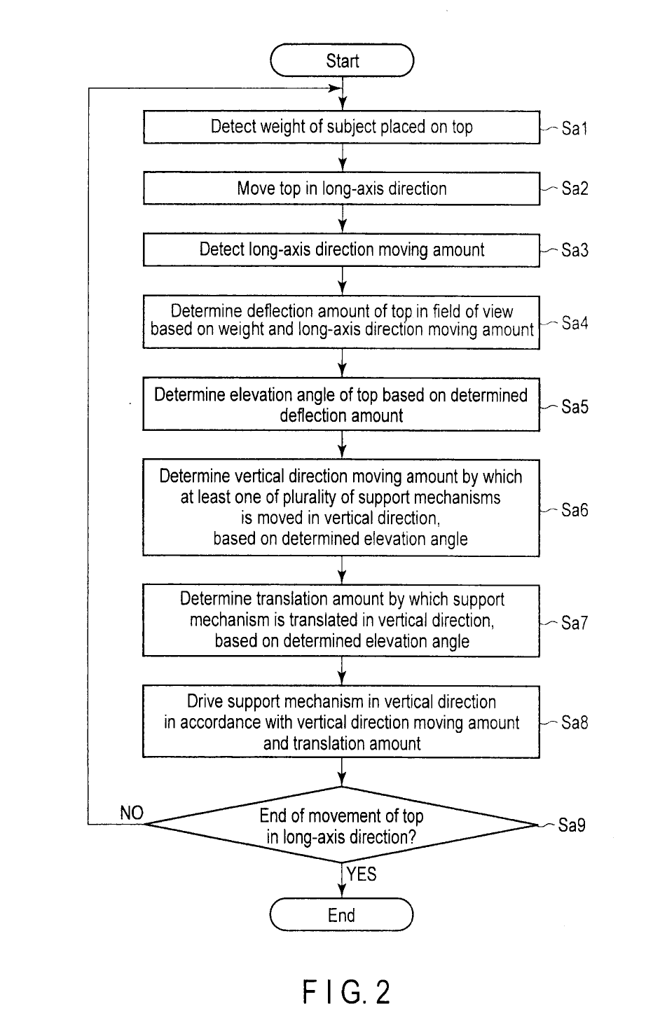

FIG. 2 is a flowchart showing an example of a procedure for vertical direction moving operation.

When a subject is placed on the top 5, the weight of the subject is detected via the support mechanisms 7 (step Sa1). Note that the apparatus may detect the weight distribution of the subject along the long-axis direction by moving the top 5, on which the subject is placed, in the long-axis direction in advance. Subsequently, the top 5 moves along the long-axis direction (step Sa2) as the rotating bodies of the long-axis direction moving mechanisms 71 rotate.

The apparatus detects the long-axis direction moving amount of the top 5 (step Sa3). The long-axis direction moving amount in step Sa3 corresponds to, for example, the feeding amount of the top 5 in S & S scanning. In helical scanning in an X-ray CT apparatus or the like, the top 5 continuously moves along the long-axis direction. For this reason, the long-axis direction moving amount coincides with, for example, about the width of the X-ray detection element in the long-axis direction or collimation width.

The apparatus determines the deflection amount of the top 5 in the FOV based on the weight and the long-axis direction moving amount (step Sa4). The apparatus determines the elevation angle of the top 5 based on the determined deflection amount (step Sa5).

The apparatus determines a vertical direction moving amount by which each of the support mechanisms 7 is moved in the vertical direction, based on the determined elevation angle (step Sa6). The apparatus determines a translation amount by which each of the support mechanisms 7 is translated in the vertical direction, based on the determined elevation angle (step Sa7). The determination unit 11 may determine a composite moving amount as a combination of a vertical direction moving amount and a translation amount by combining the processing in step Sa6 with the processing in step Sa7 based on the weight and the long-axis direction moving amount. The apparatus drives the vertical direction moving mechanism 73 of each of the support mechanisms 7 in accordance with the determined vertical direction moving amount and translation amount (step Sa8).

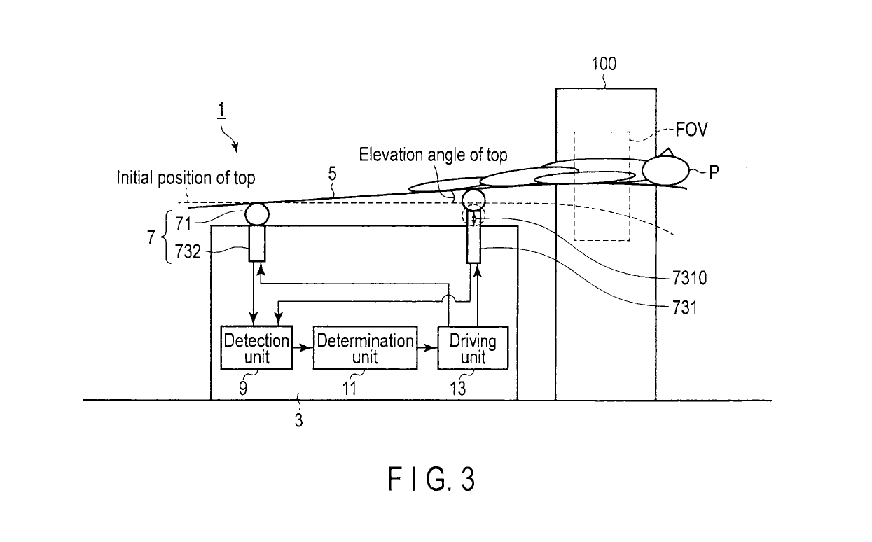

FIG. 3 is a view showing an example of driving a vertical direction moving mechanism 731 near a gantry 100 upward in the vertical direction in accordance with a vertical direction moving amount. Referring to FIG. 3, driving the vertical direction moving mechanism 731 near the gantry 100 by a vertical direction moving amount 7310 upward in the vertical direction will make part of the top 5 in the FOV become horizontal. At this time, the angle defined between the initial position of the top and the top 5 becomes the elevation angle shown in FIG. 3. Note that the apparatus may drive a vertical direction moving mechanism 732 far from the gantry 100 downward in the vertical direction instead of driving the vertical direction moving mechanism 731 near the gantry 100 upward in the vertical direction.

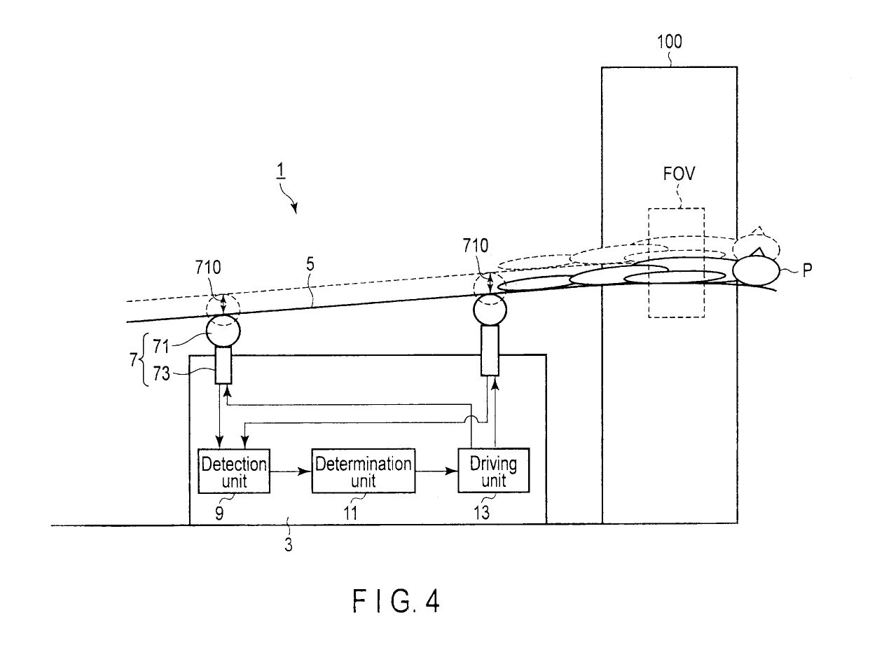

FIG. 4 is a view showing an example of translating the top 5 downward in the vertical direction in accordance with a translation amount. As shown in FIG. 4, the apparatus translates the top 5 downward in the vertical direction by a translation amount 710.

If the movement of the top 5 in the long-axis direction is not complete (step Sa9), the apparatus repeats the operation and processing in steps Sa1 to Sa8.

### (Modification)

In this modification, the medical image diagnostic apparatus bed 1 according to this embodiment is applied to an X-ray CT apparatus as an example of a medical image diagnostic apparatus. Note that the medical image diagnostic apparatus bed 1 according to the embodiment can also be applied to other types of medical image diagnostic apparatuses. Other medical image diagnostic apparatuses include, for example, an X-ray diagnostic apparatus used for angiography for a subject.

An X-ray CT apparatus 10 including the medical image diagnostic apparatus bed 1 according to this embodiment will be described below with reference to the accompanying drawings. Note that there are various types of X-ray CT apparatus 10 such as Rotate/Rotate-Type in which an X-ray tube 101 and X-ray detection unit 103 integrally rotate around a subject, and Stationary/Rotate-Type in which many X-ray detection elements arrayed in a ring shape are fixed and only an X-ray tube 101 rotates around a subject. Any type of X-ray CT apparatus is applicable to this modification. Reconstruction of an image requires projection data for 360° corresponding to one round around a subject. Even the half-scan method requires projection data for 180°+fan angle. Either reconstruction method is applicable to this modification. As for a mechanism of changing incident X-rays into charges, indirect conversion and direct conversion are the mainstream. In indirect conversion, X-rays are converted into light by a phosphor such as a scintillator, and the light is further converted into charges by a photoelectric converter such as a photodiode. Direct conversion exploits generation of electron-hole pairs in a semiconductor such as selenium by X-rays, and movement of them to an electrode, i.e., photoconductive phenomenon. As an X-ray detection element, either of these methods can be used. Recently, so-called multi-tube X-ray CT apparatuses in which a plurality of pairs each of an X-ray tube 101 and X-ray detection unit 103 are mounted on a rotating ring 102 are being commercialized, and their peripheral techniques are being developed. In this modification, both a conventional single-tube X-ray CT apparatus and multi-tube X-ray CT apparatus are applicable. Here, the single-tube type will be explained.

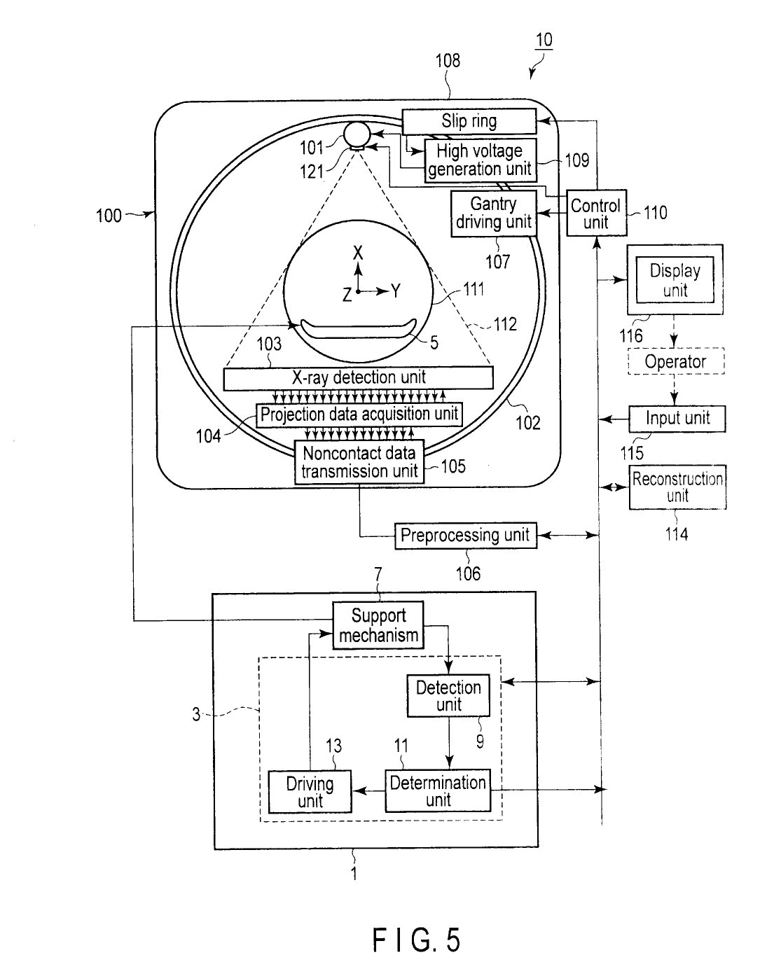

FIG. 5 is a block diagram showing an example of the arrangement of the X-ray computed tomography apparatus 10 including the medical image diagnostic apparatus bed 1. The X-ray CT apparatus 10 according to this modification includes the gantry (imaging unit) 100, a preprocessing unit 106, a reconstruction unit 114, an input unit 115, a display unit 116, a control unit 110, and the bed 1. When the medical image diagnostic apparatus bed 1 is applied to an X-ray diagnostic apparatus, the imaging unit includes, for example, a C-arm or Ω arm, an X-ray tube, and an X-ray detection unit.

The gantry unit 100 houses a rotation support mechanism. The rotation support mechanism includes a rotating ring 102, a ring support mechanism which supports the rotating ring 102 to be freely rotatable about the rotation axis Z, and a gantry driving unit 107 (motor) which drives the ring to rotate. The rotating ring 102 is equipped with the X-ray tube 101, a high voltage generation unit 109, and an area detector (to be referred to as an X-ray detection unit 103 hereinafter) which is also called a two-dimensional array type or multi-array type detector.

The high voltage generation unit 109 generates a tube voltage to be applied to the X-ray tube 101 and a tube current to be supplied to the X-ray tube 101. The high voltage generation unit 109 generates a tube voltage and a tube current in accordance with control signals input from the control unit 110 (to be described later) via a slip ring 108.

The X-ray tube 101 emits X-rays from an X-ray focus upon receiving a tube voltage and a tube current from the high voltage generation unit 109.

A collimator 121 is attached to the X-ray irradiation window on the front surface of the X-ray tube 101. The collimator 121 shapes X-rays emerging from the X-ray focus into, for example, a cone beam shape (pyramidal shape). Referring to FIG. 1, dotted lines 112 indicate an FOV. The X-axis is a straight line which is perpendicular to the rotation axis Z and extends upward in the vertical direction. The Y-axis is a straight line perpendicular to the X-axis and the rotation axis Z. For the sake of simplicity, the following description will be made on the assumption that the center of the FOV (to be referred to as the FOV center hereinafter) is located on the rotation axis.

The X-ray detection unit 103 is attached at a position and angle at which the X-ray detection unit 103 faces the X-ray tube 101 via the rotation axis Z. The X-ray detection unit 103 includes a plurality of X-ray detection elements. The following description assumes that a single X-ray detection element forms a single channel. A plurality of channels are perpendicular to the rotation axis Z and, with the focus of radiated X-rays as the center, two-dimensionally arrayed in two directions, i.e., a slice direction and the direction of an arc (channel direction) whose radius is the distance from this center to the center of the light-receiving unit of an X-ray detection element for one channel. The two-dimensional array is constructed by arranging, in the slice direction, a plurality of arrays each of a plurality of channels arrayed one-dimensionally in the channel direction. The X-ray detection unit 103 having this two-dimensional X-ray detection element array may be constructed by arranging, in the slice direction, a plurality of arrays each of a plurality of above-described modules arrayed one-dimensionally in an almost arc direction. Alternatively, the X-ray detection unit 103 may be constructed by a plurality of modules each obtained by arraying a plurality of X-ray detection elements in one line. At this time, the respective modules are arrayed one-dimensionally in an almost arc direction along the channel direction.

When performing imaging or scanning, a subject P is placed on the top 5 and inserted into a cylindrical imaging region 111 between the X-ray tube 101 and the X-ray detection unit 103. A projection data acquisition unit 104 (a so-called data acquisition circuit) called a DAS (Data Acquisition System) is connected to the output of the X-ray detection unit 103.

The projection data acquisition unit 104 is provided, for each channel, with an I-V converter which converts a current signal from each channel of the X-ray detection unit 103 into a voltage, an integrator which periodically integrates this voltage signal in synchronism with the exposure period of X-rays, an amplifier which amplifies an output signal from the integrator, and an analog/digital converter which converts an output signal from the amplifier into a digital signal. The projection data acquisition unit 104 outputs data (pure raw data) to the preprocessing unit 106 via a noncontact data transmission unit 105 using magnetic transmission/reception or optical transmission/reception. The projection data acquisition unit 104 changes the integration interval of the integrator in accordance with a scan under the control of the control unit 110 (to be described later).

The preprocessing unit 106 performs preprocessing for the pure raw data output from the projection data acquisition unit 104. Preprocessing includes, for example, sensitivity nonuniformity correction processing between channels and the processing of correcting an extreme decrease in signal intensity or signal omission due to an X-ray absorber, mainly by a metal portion. The data (called raw data or projection data; projection data in this case) output from the preprocessor 106 immediately before reconstruction processing is stored in a projection data storage unit including a magnetic disk, magneto-optical disk, or semiconductor memory in association with data (to be referred to as view angle data hereinafter) representing view angles at the time of data acquisition. That is, the preprocessing unit 106 generates projection data for each of a plurality of channels in association with view angle data.

Note that projection data is a set of data values corresponding to the intensities of X-rays transmitted through a subject. For the sake of descriptive convenience, assume that a set of projection data acquired nearly at the same time with one shot at the same view angle throughout all the channels will be referred to as a projection data set. The respective view angles are represented by angles in the range of 00 to 360° which represent the respective positions on a circular orbit centered on the rotation axis Z, along which the X-ray tube 101 revolves, with the angle of the uppermost portion on the circular orbit in an upward vertical direction from the rotation axis Z being 00. Note that projection data of a projection data set which corresponds to each channel is identified by a view angle, cone angle, and channel number.

The reconstruction unit 114 has a function of reconstructing an almost columnar three-dimensional image (volume data) regarding a reconstruction region by a feldkamp method or cone beam reconstruction method based on a projection data set for which the view angle falls within the range of 360° or 180°+fan angle. The reconstruction unit 114 has a function of reconstructing a two-dimensional image (tomographic image) by, e.g., a fan beam reconstruction method (also called a fan beam convolution back projection method) or a filtered back projection method. The feldkamp method is a reconstruction method used when projection rays cross each other with respect to the reconstruction plane, similar to cone beams. The feldkamp method is an approximate image reconstruction method in which rays are regarded as fan projection beams in convolution on the premise that the cone angle is small, and back projection is processed along rays in scanning. The cone beam reconstruction method is a reconstruction method of correcting projection data in accordance with the angle of a ray with respect to the reconstruction plane as a method capable of suppressing a cone angle error compared to the feldkamp method. The reconstruction unit 114 selects a projection data set used for reconstruction in accordance with a scan under the control of the control unit 110 (to be described later).

An interface (not shown) connects the X-ray computed tomography apparatus 10 to an electronic communication line (to be referred to as a network hereinafter). A radiology department information management system (not shown), a hospital information system (not shown), and the like are connected to the network.

The display unit 116 displays the medical image reconstructed by the reconstruction unit 114, conditions set for X-ray computed tomography, and the like.

The input unit 115 inputs, to the X-ray computed tomography apparatus 10, various types of instructions, commands, information, selections, and settings from the operator. The input various types of instructions, commands, information, selections, and settings are output to the control unit 110 and the like. The input unit 115 includes a trackball, switch buttons, mouse, a keyboard, and the like (none of which are shown) for setting a region of interest (ROI) and the like. The input unit 115 may be provided on a gantry cover covering the gantry 100. The input unit 115 inputs information indicating operation concerning the movement of the top 5 on the bed 1 (to be described later). More specifically, the input unit 115 inputs information indicating the moving direction and moving velocity of the top 5 along the long-axis direction and vertical direction, the operation of enabling/disabling the movement of the top 5, and the like. The input unit 115 inputs the number of steps, a step width, and the like in S & S scanning.

The input unit 115 detects the coordinate point of a cursor displayed on the display screen, and outputs the detected coordinate point to the control unit 110. Note that the input unit 115 may be a touch panel arranged to cover the display screen. In this case, the input unit 115 detects a touched and indicated coordinate point based on a coordinate reading principle such as an electromagnetic induction method, electromagnetic distortion method, or pressure sensitive method, and outputs the detected coordinate point to the control unit 110.

The control unit 110 functions as the main unit of an X-ray computed tomography apparatus 10. The control unit 110 includes a CPU and a memory (neither of which is shown). The control unit 110 controls the high voltage generation unit 109 for X-ray computed tomography, the gantry 100, and the like based on examination schedule data and control programs stored in the memory (not shown). More specifically, the control unit 110 temporarily stores, in a memory (not shown), instructions and the like input by the operator and sent from the input unit 115 (to be described later), a radiology department information management system (not shown), a hospital information system (not shown), and the like. The control unit 110 controls the high voltage generation unit 109, the gantry 100, and the like based on these pieces of information temporarily stored in the memory. The control unit 110 reads out control programs for executing predetermined image generation/display processing and the like from a storage unit (not shown), expands the programs in its own memory, and executes computation, processing, and the like associated with various types of processes.

The bed 1 includes the bed main body 3, the top 5, and the plurality of support mechanisms 7 which support the top 5 at a plurality of corresponding fulcrums. The bed main body 3 includes the detection unit 9, the determination unit 11, and the driving unit 13. The support mechanism 7 includes the long-axis direction moving mechanism 71 which supports the top 5 with movability of the top 5 in the long-axis direction of the top 5 and the vertical direction moving mechanism 73 which supports the top 5 and the long-axis direction moving mechanism 71 with movability of the top 5 in the vertical direction of the top 5. Note that the medical image diagnostic apparatus bed 1 may be provided for an X-ray diagnostic apparatus.

A subject is placed on the top 5. The plurality of support mechanisms 7 respectively having a plurality of fulcrums support the top 5. The long-axis direction moving mechanisms 71 (to be described later) support the top 5 with movability in the long-axis direction. The vertical direction moving mechanisms 73 (to be described above) support the top 5 with movability in the vertical direction through the long-axis direction moving mechanisms 71.

Each of the support mechanisms 7 has a fulcrum which supports the top 5. Each of the support mechanisms 7 includes the long-axis direction moving mechanism 71 and the vertical direction moving mechanism 73. The long-axis direction moving mechanisms 71 supports the top 5 with its movability in the long-axis direction. The vertical direction moving mechanisms 73 supports the long-axis direction moving mechanisms 71 with their movability in the vertical direction.

The detection unit 9 detects the weight of the subject placed on the top 5 through the support mechanisms 7. The detection unit 9 detects the long-axis direction moving amount of the top 5. The detection unit 9 outputs the detected weight and long-axis direction moving amount to the determination unit 11 (to be described later). More specifically, the detection unit 9 detects a weight applied to each of the support mechanisms 7 in accordance with the movement of the top 5, on which the subject is placed, in the long-axis direction. Note that in helical scanning, the top 5 continuously moves along the long-axis direction. For this reason, the detection unit 9 detects a long-axis direction moving amount for each nearly the width of an X-ray detection element in the long-axis direction or collimation width. Note that the detection unit 9 can continuously detect long-axis direction moving amounts throughout the execution of helical scanning.

The determination unit 11 determines the deflection amount of the top 5 based on the long-axis direction moving amount and weight output from the detection unit 9. Note that the determination unit 11 may determine a deflection amount based on a total long-axis direction moving amount and a weight in S & S scanning. The determination unit 11 determines the elevation angle of the top 5 based on the deflection amount. The determination unit 11 determines an elevation angle so as to make part of the top 5 moved in the long-axis direction in the FOV become horizontal. The determination unit 11 determines a vertical direction moving amount for at least one of the plurality of support mechanisms 7 based on the determined elevation angle. The determination unit 11 outputs the vertical direction moving amount for each of the plurality of support mechanisms 7 to the driving unit 13 (to be described later).

The determination unit 11 determines a translation amount by which each of the plurality of support mechanisms 7 is translated along the vertical direction, based on the determined elevation angle. The determination unit 11 determines a translation amount so as to place the top 5 at a predetermined position in the FOV in each step in S & S scanning.

The driving unit 13 drives the support mechanisms 7. More specifically, for example, the driving unit 13 drives the long-axis direction moving mechanisms 71 to feed the top 5 along the long-axis direction in each of a plurality of steps in S & S scanning with a predetermined step width. With this driving operation, the top 5 moves along the long-axis direction with a predetermined step width. The driving unit 13 drives the vertical direction moving mechanisms 73 of the support mechanisms 7 in each step in accordance with the vertical direction moving amount output from the determination unit 11. With this driving operation, part of the top 5 in the FOV becomes horizontal.

The driving unit 13 drives the plurality of vertical direction moving mechanisms 73 to translate the vertical direction moving mechanisms 73 of the support mechanisms 7 along the vertical direction based on the translation amount output from the determination unit 11. With this driving operation, part of the top 5 in the FOV always remains the same height.

### (Vertical Direction Moving Function)

The vertical direction moving function is a function of moving the vertical direction moving mechanisms 73 in the vertical direction based on the vertical direction moving amount and translation amount determined by the determination unit 11. For the sake of simplicity, operation concerning the vertical direction moving function (to be referred to as vertical direction moving operation hereinafter) in each step in S & S scanning will be described below. Note that it is also possible to apply vertical direction moving operation to the beds of other medical image diagnostic apparatuses. In addition, it is possible to apply vertical direction moving operation to scanning other than S & S scanning, e.g., continuous top moving imaging (to be referred to as helical scanning hereinafter).

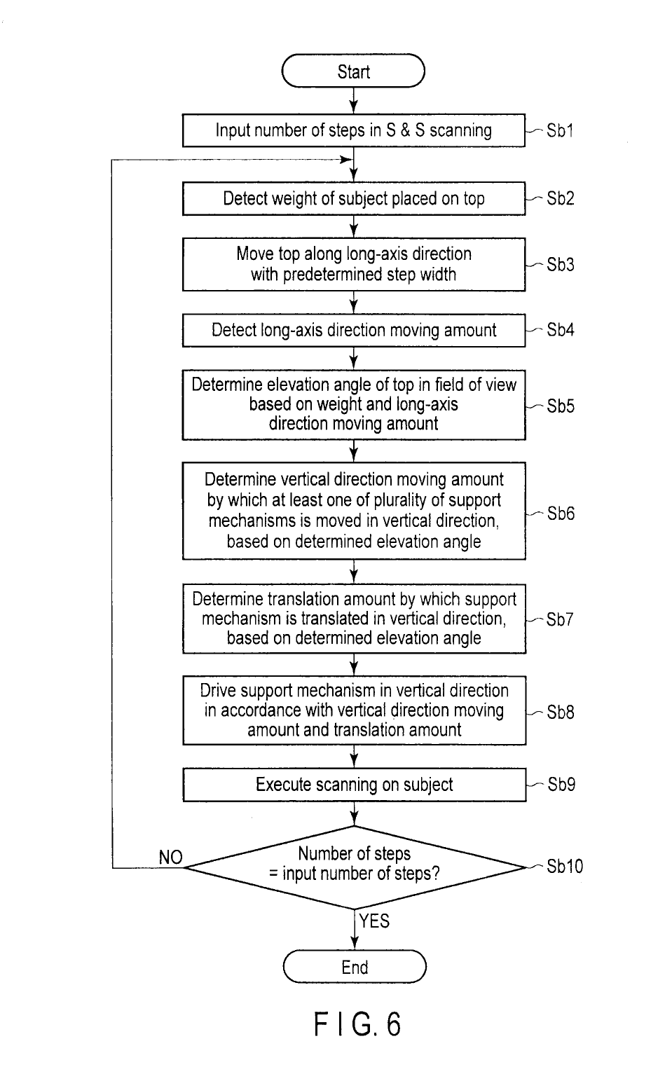

FIG. 6 is a flowchart showing an example of a procedure for vertical direction moving operation.

In S & S scanning, the apparatus inputs the number of times of step movement of the top 5 (step Sb1). When a subject is placed on the top 5, the apparatus detects the weight of the subject via the support mechanisms 7 (step Sb2). The long-axis direction moving mechanisms 71 moves the top 5 along the long-axis direction with a predetermined step width (step Sb3).

The apparatus detects the long-axis direction moving amount of the top 5 (step Sb4). The long-axis direction moving amount in step Sb4 corresponds to the feeding amount of the top 5 in S & S scanning. The apparatus determines the elevation angle of the top 5 in the FOV based on the weight and the long-axis direction moving amount (step Sb5). The apparatus determines a vertical direction moving amount by which each of the support mechanisms 7 is moved in the vertical direction, based on the determined elevation angle (step Sb6). The apparatus determines a translation amount by which the support mechanisms 7 and the top 5 are translated in the vertical direction, based on the determined elevation angle (step Sb7). The apparatus drives the vertical direction moving mechanisms 73 of the support mechanisms 7 in accordance with the determined vertical direction moving amount and the translation amount (step Sb8).

The operation in step Sb8 makes the top become horizontal in the FOV. In addition, the apparatus moves the top 5 to the same level in each step. The apparatus executes scanning on the subject (step Sb9). The apparatus repeats the operation in steps Sb2 to Sb9 until the number of steps coincides with the input number of steps (step Sb10).

According to the above arrangement, the following effects can be obtained.

The medical image diagnostic apparatus bed 1 according to this embodiment can actively change the height of the top 5 based on the long-axis direction moving amount and weight detected by the detection unit 9. That is, the medical image diagnostic apparatus bed 1 can move the top 5 to the elevation angle determined based on the long-axis direction moving amount and the weight. Tilting the top 5 at the determined elevation angle can eliminate top sag (the deflection amount of the top) corresponding to the long-axis direction moving amount of the top 5 in the FOV. This makes it possible to eliminate top sag independently of the rigidity of the top 5.

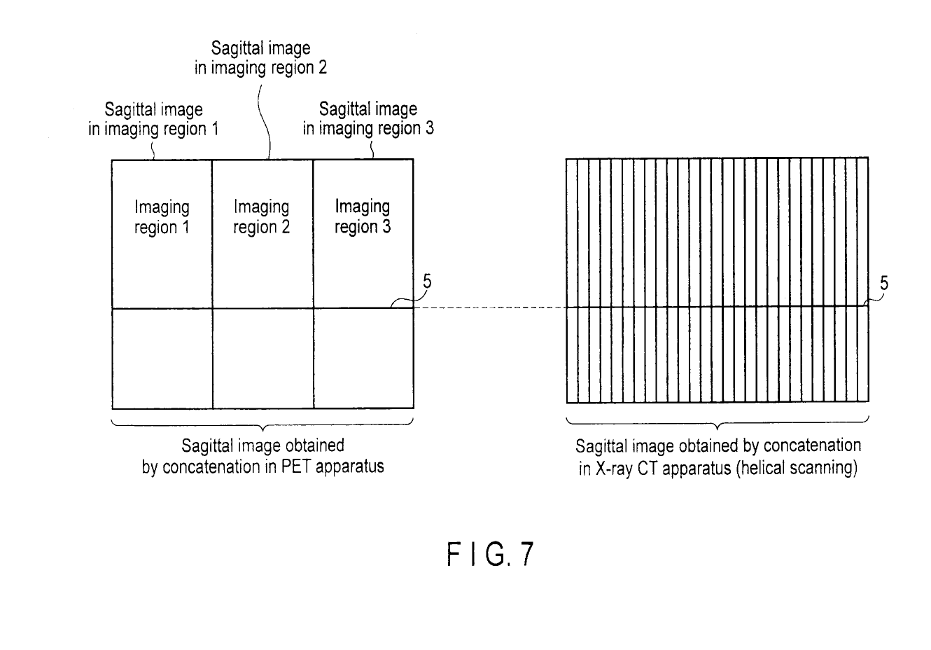

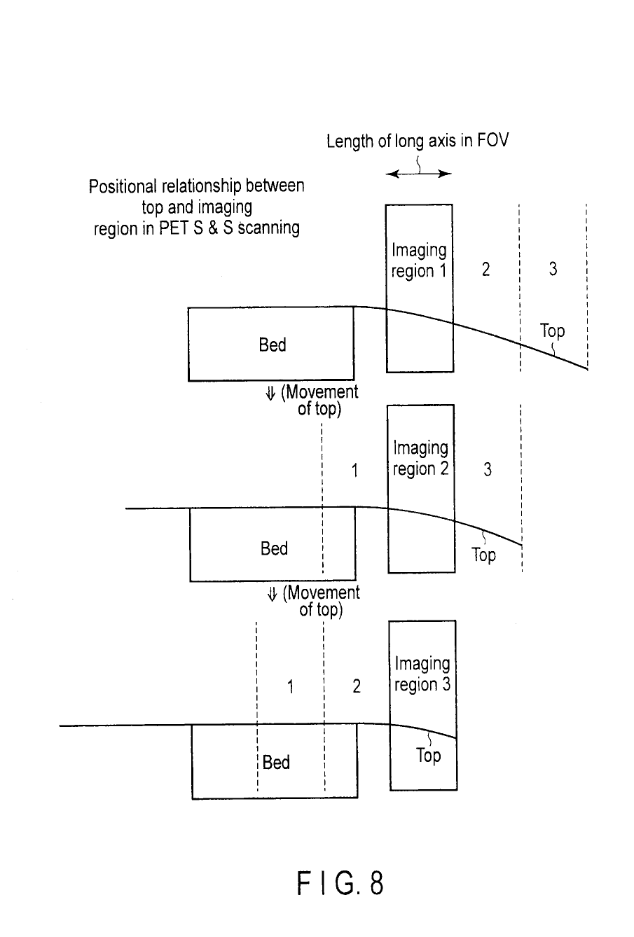

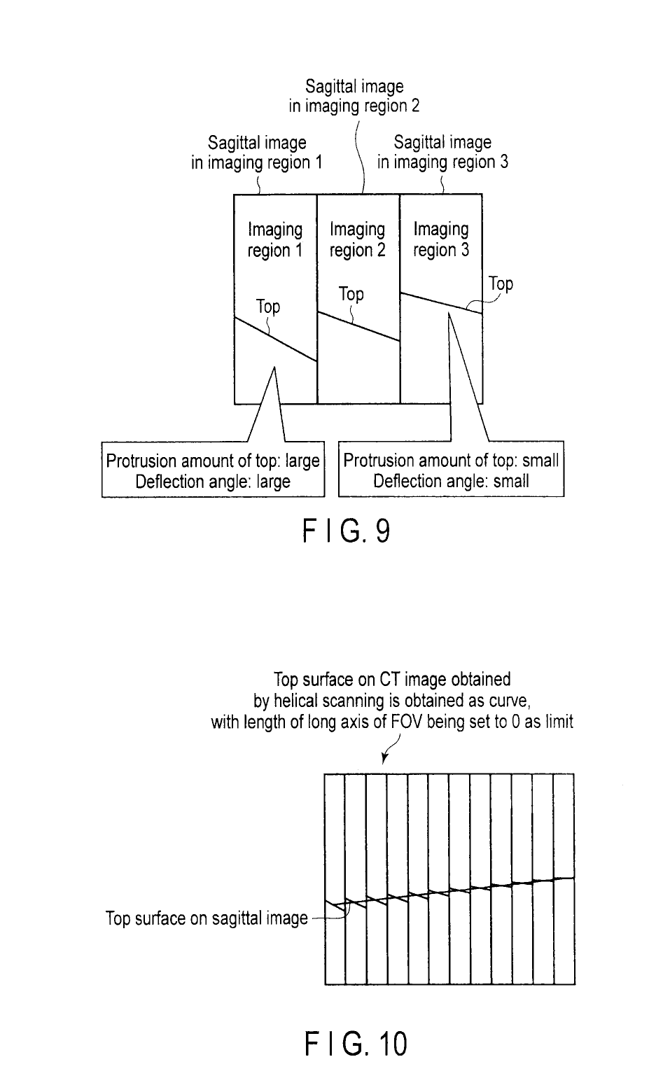

In addition, the X-ray CT apparatus 10 including the medical image diagnostic apparatus bed 1 according to the modification of this embodiment can keep the top 5 horizontal in the FOV and can also keep the position of the top surface (the height from the bottom surface of the bed to the top surface) constant. FIG. 7 is a view showing an example of the image obtained by concatenating the reconstructed images reconstructed from a plurality of imaging regions to each other along the long-axis direction in a PET apparatus and an X-ray CT apparatus according to the modification of the embodiment. As shown in FIG. 7, at is possible to execute scanning by the X-ray CT apparatus 10 in accordance with the position of the top 5 on a sagittal image in a PET apparatus.

This makes it possible to generate medical images for more accurate radiotherapy plans. In addition, for example, it is possible to easily execute positioning between the sagittal image reconstructed based on helical scanning in the X-ray CT apparatus 10 including the medical image diagnostic apparatus bed 1 and the sagittal image reconstructed based on S & S scanning in a PET apparatus including the medical image diagnostic apparatus bed 1. This can therefore solve the problem of positioning due to top sag. In addition, it is possible to perform positioning in accordance with position (height) of the radiotherapy top. According to the above description, it is possible to make more accurate radiotherapy plans.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical image diagnostic apparatus bed comprising:
a top on which a subject is placed;
a support mechanism configured to support the top with adjustability of an elevation angle of the top;
a determination unit configured to determine the elevation angle based on information concerning deflection of the top; and
a driving unit configured to drive the support mechanism in accordance with the determined elevation angle.

2. The medical image diagnostic apparatus bed according to claim 1, wherein the information concerning the deflection includes at least one of a long-axis direction moving amount by which the top is moved in a long-axis direction of the top, a weight of the subject on the top, and a deflection amount of the top.

3. The medical image diagnostic apparatus bed according to claim 2, further comprising a detection unit configured to detect at least one of the long-axis direction moving amount, the weight, and the deflection amount.

4. The medical image diagnostic apparatus bed according to claim 1, wherein the support mechanism is configured to support the top with movability of the top in a vertical direction,
the determination unit is configured to determine a translation amount by which the top is translated along the vertical direction, based on the elevation angle, and
the driving unit is configured to drive the support mechanism in the vertical direction based on the determined translation amount.

5. The medical image diagnostic apparatus bed according to claim 2, wherein the determination unit is configured to determine the elevation angle based on a distribution of the weights and the long-axis direction moving amount.

6. The medical image diagnostic apparatus bed according to claim 1, wherein the determination unit is configured to
determine the weight distribution along a long-axis direction of the top based on a weight of the subject and a weight of the subject placed on the top, and
determine the elevation angle based on the weight distribution and a long-axis direction moving amount of the top.

7. The medical image diagnostic apparatus bed according to claim 1, wherein the support mechanism is configured to support the top at a plurality of fulcrums with movability of the top in a long-axis direction of the top and a vertical direction,
the determination unit is configured to determine a vertical direction moving amount by which the support mechanism is moved along the vertical direction, based on the elevation angle, and
the driving unit is configured to drive the support mechanism in accordance with the determined vertical direction moving amount.

8. The medical image diagnostic apparatus bed according to claim 3, wherein the determination unit is configured to
calculate a moment of the top with respect to a fulcrum which supports the top, based on the long-axis direction moving amount and the weight,
calculate the deflection based on the calculated moment, and
determine the elevation angle based on the calculated deflection.

9. The medical image diagnostic apparatus bed according to claim 1, wherein the determination unit is configured to
estimate a deflection amount of the top based on patient information about the subject and an imaging plan for the subject, and
determine the elevation angle based on the estimated deflection amount of the top.

10. A medical image diagnostic apparatus comprising:
an imaging unit configured to image or scan a subject placed on a top;
a support mechanism configured to support the top with adjustability of an elevation angle of the top;
a determination unit configured to determine the elevation angle based on information concerning deflection of the top; and
a driving unit configured to drive the support mechanism in accordance with the determined elevation angle.

11. The medical image diagnostic apparatus according to claim 10, wherein the information concerning the deflection includes at least one of a long-axis direction moving amount by which the top is moved in a long-axis direction of the top, a weight of the subject on the top, and a deflection amount of the top.

12. The medical image diagnostic apparatus according to claim 11, further comprising a detection unit configured to detect at least one of the long-axis direction moving amount, the weight, and the deflection amount.

13. The medical image diagnostic apparatus according to claim 10, wherein the support mechanism is configured to support the top with movability of the top in a vertical direction,
the determination unit is configured to determine a translation amount by which the top is translated along the vertical direction, based on the elevation angle, and
the driving unit is configured to drive the support mechanism in the vertical direction based on the determined translation amount.

14. The medical image diagnostic apparatus according to claim 11, wherein the determination unit is configured to determine the elevation angle based on a distribution of the weights and the long-axis direction moving amount.

15. The medical image diagnostic apparatus according to claim 10, wherein the determination unit is configured to
determine the weight distribution along a long-axis direction of the top based on a weight of the subject and a weight of the subject placed on the top, and
determine the elevation angle based on the weight distribution and a long-axis direction moving amount of the top.

16. The medical image diagnostic apparatus according to claim 10, wherein the support mechanism is configured to support the top at a plurality of fulcrums with movability of the top in a long-axis direction of the top and a vertical direction,
the determination unit is configured to determine a vertical direction moving amount by which the support mechanism is moved along the vertical direction, based on the elevation angle, and
the driving unit is configured to drive the support mechanism in accordance with the determined vertical direction moving amount.

17. The medical image diagnostic apparatus according to claim 12, wherein the determination unit is configured to
calculate a moment of the top with respect to a fulcrum which supports the top, based on the long-axis direction moving amount and the weight, and
determine the elevation angle based on the calculated moment.

18. The medical image diagnostic apparatus according to claim 10, wherein the determination unit is configured to
estimate a deflection amount of the top based on patient information about the subject and an imaging plan for the subject, and
determine the elevation angle based on the estimated deflection amount of the top.
